(19) United States
(12) Patent Application Publication
(10) Pub. No.: US 2023/0124906 A1
(43) Pub. Date: Apr. 20, 2023

(54) ENDOSCOPE-ATTACHED PERCUTANEOUS GASTROSTOMY TUBE

(71) Applicant: ALPINE MEDICAL DEVICES, LLC, Carmel, CA (US)

(72) Inventor: Jan P. KAMLER, Carmel, CA (US)

(73) Assignee: ALPINE MEDICAL DEVICES, LLC, Carmel, CA (US)

(21) Appl. No.: 17/792,433

(22) PCT Filed: Jan. 15, 2021
(86) PCT No.: PCT/US21/13749

§ 371 Date: Jul. 13, 2022

Related U.S. Application Data
(60) Provisional application No. 62/962,118, filed on Jan. 16, 2020.

(51) Int. Cl. A61B 1/00

(52) U.S. Cl. CPC A61B 1/00135; A61B 1/00154

(57) ABSTRACT

A gastronomy tube includes an elongate tubular portion, a stop, and at least one wire loop. The elongate tubular portion is configured to extend along a length of an endoscope and to reversibly attach to the endoscope. The stop is at a proximal end of the elongate tubular portion. The at least one wire loop is at a distal end of the elongate tubular portion and is configured to extend distally from a distal end of the endoscope when the elongate tubular portion is attached to the endoscope.

### CROSS REFERENCE TO RELATED APPLICATIONS

This application claims priority to U.S. Provisional Patent Application No. 62/962,118, filed on Jan. 16, 2020, titled “OVER THE SCOPE PERCUTANEOUS ENDOSCOPIC GASTROSTOMY TUBE,” the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

A percutaneous endoscopic gastrostomy (or feeding) tube is a tube that is passed into a patient's stomach through the abdominal wall to provide means of feeding when oral intake is not adequate. While endoscopic placement of the feeding tube by an endoscopist is the most common method of delivery, it can also be placed by a surgeon or an interventional radiologist.

Currently available endoscopic gastrostomy feeding tubes, commonly made from medical grade silicone and 20F in diameter, have been delivered to patients percutaneously in substantially the same way for more than 30 years. A typical method is shown in FIGS. 1A-1F. As shown in FIG. 1A, during percutaneous placement of a typical feeding tube, a scope 100 is inserted into the stomach 101, and the stomach 101 is distended with air.

Transillumination of the abdominal wall 114 is performed from inside of the stomach 101. The area of maximum transillumination on the outer surface of the abdominal wall 114 is marked and disinfected. Lidocaine and a small-bore needle 102 are used for local anesthesia. As shown in FIG. 1B, a large bore needle 103 is then inserted through the abdominal wall 114, through the gastric wall 113, and inside the lumen of the stomach 101. A long wire 104 is fed through the large bore needle 103 into the stomach 101. As shown in FIG. 1C, the wire 104 is then grasped with a snare 105 that has been inserted into the stomach 101 though the biopsy channel of the scope 100. As shown in FIG. 1D, the wire 104 is pulled proximally through the patient's mouth 111 using the snare 105 so that one end 106 is sticking out though the abdominal wall 114 and the other end 107 from the mouth 111. As shown in FIG. 1E, a long gastrostomy feeding tube 108 with a sharp, firm front tip 109 and with a soft stop knob 110 at the distal end is attached to the end 107 of the wire 104 that is protruding from the mouth 111. Referring to FIG. 1F, the feeding tube 108 is then pulled into the stomach 101 by pulling the end 106 of the wire 104 protruding from the abdominal wall 114. Finally, the feeding tube 108 can be pulled through the gastric wall 113 and the abdominal wall 114. The sharp front tip 109 can be used to cut through the walls 113, 114. The feeding tube 108 is then secured to the abdominal wall 114 by an external bolster 115 with the knob 110 left inside the stomach 101. The long end of the tube 108 is cut and a Y-port feeding adaptor 116 is attached such that food can be provided directly to the stomach 101 via the adaptor 116.

The typical procedure for delivering a feeding tube takes a fairly long time (e.g., at least 30 minutes) and requires an assistant or nurse to hold the scope 100 in addition to the doctor or clinician placing the feeding tube. Moreover, currently available feeding tubes are expensive, bulky, and contain multiple parts. Accordingly, a feeding tube that solves some or all of these problems is desired.

### SUMMARY OF THE DISCLOSURE

In general, in one embodiment a gastronomy tube includes an elongate tubular portion, a stop, and at least one wire loop. The elongate tubular portion is configured to extend along a length of an endoscope and to reversibly attach to the endoscope. The stop is at a proximal end of the elongate tubular portion. The at least one wire loop is at a distal end of the elongate tubular portion and is configured to extend distally from a distal end of the endoscope when the elongate tubular portion is attached to the endoscope.

This and other embodiments can include one or more of the following features. The elongate tubular portion can include a stretchable tube configured to stretch around a circumference of the endoscope. The stretchable tube can be configured to slide axially along the endoscope when a distal force is placed on the at least one wire loop. The stretchable tube can be configured to return to its original diameter as the stretchable tube is slid off of the endoscope. The gastronomy tube can further include a plurality of triangular spikes extending distally from the elongate tubular portion. The at least one wire loop can be attached to the plurality of triangular spikes and extend distally therefrom. The triangular spikes can include a harder material than the stretchable tube. The triangular spikes can be configured to converge into a cone shape as the stretchable tube is slid distally off of the endoscope. The gastronomy tube can further include a mesh tubing extending distally from the elongate tubular portion. The at least one wire loop can be attached to the mesh tubing and extend distally therefrom. The mesh tubing can be configured to converge into a cone shape as the stretchable tube is slid distally off of the endoscope. The at least one wire loop can be 8-15 cm long. The at least one wire loop can include a wire having a diameter of less than 1 mm. The elongate tubular portion can be configured to run alongside and parallel to the endoscope. The gastronomy tube can further include a tapered section that tapers in diameter from the elongate tubular portion towards the at least one wire loop. The gastronomy tube can further include a distal attachment mechanism configured to reversibly attach the elongate tubular portion to the endoscope by engaging with the tapered section. The distal attachment mechanism can include an annular ring configured to fit over a distal tip of the endoscope and to hold the tapered section therein. The distal attachment mechanism can further include a plurality of teeth, perforations, or flaps configured to release the tapered section from the endoscope upon lateral movement of the at least one wire loop. The stop can be a soft knob. The stop can be an inflatable balloon.

In general, in one embodiment, a method of delivering a gastronomy tube includes: (1) attaching a gastronomy tube to an endoscope, (2) passing the endoscope and attached gastronomy tube through an oral cavity and into a stomach, (3) inserting a large bore needle through an abdominal wall, a gastric wall, and into a lumen of the stomach, (4) grabbing a distal loop of the gastronomy tube with the large bore needle, (5) pulling the distal loop distally such that the gastronomy tube is pulled distally out of the stomach, and (6) attaching the gastronomy tube to the abdominal wall and the gastric wall.

This and other embodiments can include one or more of the following features. Pulling the distal loop distally can disengage the gastronomy tube from the endoscope. Attaching the gastronomy tube to the endoscope can include stretching an elongate tube over a distal end of the endoscope. Pulling the distal loop distally can further cause triangular spikes of the gastronomy tube to form into a sharp conical tip cutting through the abdominal wall and gastric wall as the gastronomy tube is pulled distally out of the stomach. The triangular spikes can be radially flush with a distal end of the endoscope while the endoscope and attached gastronomy tube are passed through the oral cavity and into the stomach. Attaching the gastronomy tube to the endoscope can include placing an annular attachment mechanism around a distal tip of the endoscope and extending the gastronomy tube parallel to the endoscope. The method can further include disengaging the gastronomy tube from the endoscope by pulling laterally on the distal loop. The endoscope can be left in the stomach during the step of pulling the distal loop distally. The method can further include removing the endoscope from the stomach prior to the step of pulling the distal loop distally. The method can further include cutting a distal end of the gastronomy tube after the gastronomy tube is pulled distally out of the stomach. The time to complete the steps of passing the endoscope, inserting the large bore needle, grabbing the distal loop, pulling the distal loop, and attaching the gastronomy tube can be less than 30 minutes. The large bore needle can include an inner stylet and an outer sheath. Grabbing the distal loop of the gastronomy tube with the large bore needle can include grabbing the distal loop with a groove in the inner stylet. The method can further include pushing the outer sheath over the distal loop when the distal loop is positioned in the groove prior to the step of pulling the distal loop distally. Attaching the gastronomy tube to the abdominal wall and the gastric wall can include attaching the gastronomy tube with an external bolster. Attaching the gastronomy tube with the external bolster can include sliding the gastronomy tube through a lateral slit in the external bolster to a central bore in the bolster and then locking the slit to fasten the external bolster around the gastronomy tube.

In general, in one embodiment, a large bore needle includes an inner stylet and an outer sheath. The inner stylet has a central longitudinal axis and a sharp distal tip. The inner stylet further has a groove in an outer surface thereof extending transverse to the central longitudinal axis and configured to hold a wire therein. The outer sheath is positioned over the inner stylet and is configured to slide over and reversibly lock the wire inside of the groove.

This and other embodiments can include one or more of the following features. The inner stylet can further include a plurality of channels extending from the groove towards the sharp distal tip, and the plurality of channels can be configured to hold the wire therein. The plurality of channels can extend parallel to the central longitudinal axis. The outer sheath can further include a plurality of axial channels on an inner circumference thereof configured to align with the channels of the inner stylet to hold the wire therein. The needle can include a metal, and the sheath can include a plastic.

In general, in one embodiment, a bolster for attaching a feeding tube to a patient includes a housing, a bore through the housing, a slit through the housing extending from a lateral surface of the housing to the bore, and a locking mechanism configured to lock the slit together when a feeding tube is positioned within the bore.

This and other embodiments can include one or more of the following features. The bolster can further include a core within the housing having a harder material than the housing. The core can include the locking mechanism such that the locking mechanism is positioned within the housing. The locking mechanism can be positioned external to the housing.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1F show a typical method of delivering a long feeding tube. FIG. 1A shows a scope inserted in the stomach with a small needle in the abdominal wall. FIG. 1B shows a scope in the stomach with a large bore needle and a long wire inside the stomach. FIG. 1C shows a scope in the stomach with a snare grabbing a long wire inserted through a large bore needle inserted in the stomach. FIG. 1D shows a scope out of the body, a snare through the scope grabbing a long wire which was inserted through a large bore needle, with the needle inserted in the stomach through the abdominal wall. FIG. 1E shows a long feeding tube attached to a long wire that is inserted through mouth, esophagus, stomach and the abdominal wall. FIG. 1F shows a final position of feeding tube through the abdominal wall.

FIGS. 2A-2C show an over the scope feeding tube as described herein. FIG. 2A shows the feeding tube in a retracted position relative to the scope. FIG. 2B shows the feeding tube in a partially extended position. FIG. 2C show the feeding tube in a fully extended position.

FIGS. 3A-3D show a method of delivering the feeding tube of FIGS. 2A-2C. FIG. 3A shows the feeding tube inserted into the stomach over the scope. FIG. 3B shows a large bore needle inserted into the stomach to grab the feeding tube. FIG. 3C is a close-up of FIG. 3B. FIG. 3D shows attachment of the feeding tube to the gastric and abdominal walls.

FIGS. 4A-4C show close-ups of the distal end of the feeding tube of FIGS. 2A-2C in the retracted position.

FIGS. 5A-5C show close-ups of the distal end of the feeding tube of FIGS. 2A-2C in the partially extended position.

FIGS. 6A-6C show close-ups of the distal end of the feeding tube of FIGS. 3A-3C in the fully extended position.

FIGS. 7A-7D show another exemplary over the scope feeding tube as described herein. FIG. 7A shows the feeding tube in the retracted position. FIG. 7B is a close-up of FIG. 7A. FIG. 7C shows the feeding tube in the fully extended position. FIG. 7D shows a close-up of FIG. 7C.

FIGS. 8A-8B show another exemplary over the scope feeding tube as described herein. FIG. 8A shows the feeding tube in the retracted position. FIG. 8B shows the feeding tube in the extended position.

FIGS. 9A-9F show an exemplary feeding tube configured to be attached alongside a scope as described herein. FIG. 9A shows the feeding tube attached to the scope. FIG. 9B shows the feeding tube. FIG. 9C shows a distal tapered section of the feeding tube attached to the scope. FIG. 9D is a top view of FIG. 9C. FIG. 9E shows exemplary dimensions (in mm) of the feeding tube. FIG. 9F is a detailed view of the distal portion of FIG. 9E.

FIGS. 10A-10B show another exemplary feeding tube configured to be attached alongside a scope as described herein. FIG. 10A shows the feeding tube attached to the scope.

FIG. 10B shows exemplary dimensions (in mm) of the feeding tube.

FIG. 11 shows an exemplary attachment mechanism for attaching a feeding tube to a scope as described herein.

FIGS. 12A-12B show another exemplary attachment mechanism for attaching a feeding tube to a scope as described herein. FIG. 12A shows the attachment mechanism closed. FIG. 12B shows the attachment mechanism partially open.

FIGS. 13A-13B show another exemplary attachment mechanism for attaching a feeding tube to a scope as descried herein. FIG. 13A shows the attachment mechanism closed.

FIG. 13B shows the attachment mechanism open.

FIG. 14 shows an exemplary method of delivering a feeding tube that is attached alongside a scope as described herein.

FIGS. 15A-15C shows another exemplary feeding tube configured to be attached alongside a scope as described herein. FIG. 15A shows a short version of the feeding tube.

FIG. 15B shows a long version of the feeding tube. FIG. 15C shows the feeding tube as it would appear after implantation (e.g., connected to an adaptor with the balloon inflated).

FIGS. 16A-16C show an exemplary large bore needle as described herein. FIG. 16A shows the large bore needle with the outer sheath pulled back to expose a groove in the inner stylet. FIG. 16B shows the outer sheath pushed forward to cover the groove. FIG. 16C shows the outer sheath.

FIGS. 17A-17B show another exemplary large bore needle as described herein.

FIG. 17A shows the large bore needle with a sharp inner stylet. FIG. 17B shows the large bore needle with a stylet hook.

FIGS. 18A-18C show various view of a proximal attachment mechanism for attaching a long feeding tube alongside a scope as described herein.

FIGS. 19A-19D show an exemplary external bolster as described herein. FIG. 19A shows an isometric view. FIG. 19B shows a top view. FIGS. 19C and 19D show the core of the bolster.

FIG. 20 shows another exemplary external bolster as described herein.

### DETAILED DESCRIPTION

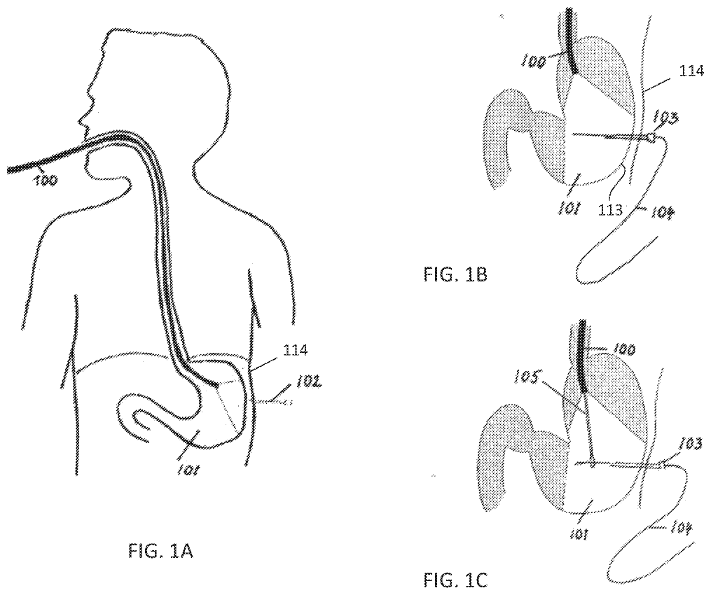

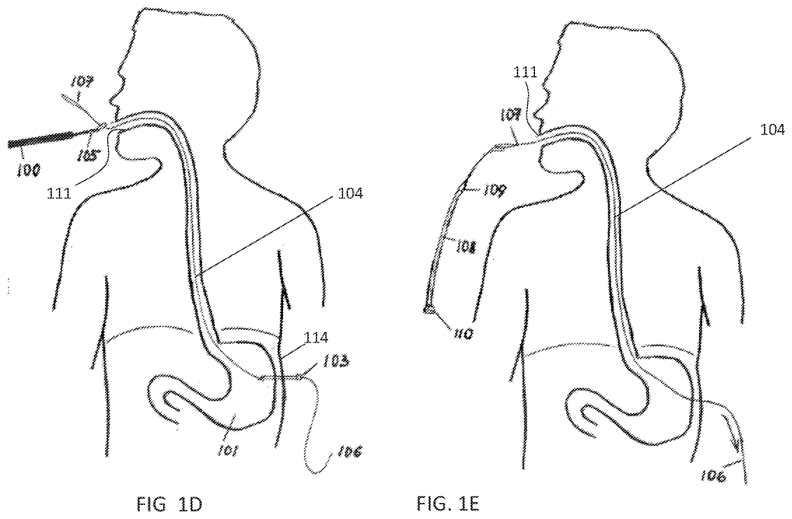

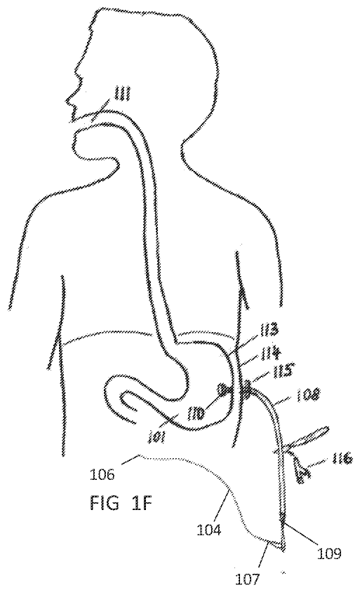

Endoscope-attached (e.g., alongside or over the scope) percutaneous gastrostomy (feeding) tubes are described herein. The feeding tubes described herein can advantageously eliminate a substantial amount (e.g., 50% or more) of the time and/or steps associated with standard percutaneous placement of feeding tubes (described with respect to FIGS. 1A-1F). For example, the feeding tubes described herein can eliminate the wires and snares, can eliminate the need for a medical assistant or nurse to hold the scope, and/or can shorten the procedural delivery time to under 30 minutes, such as under 20 minutes, such as under 15 minutes.

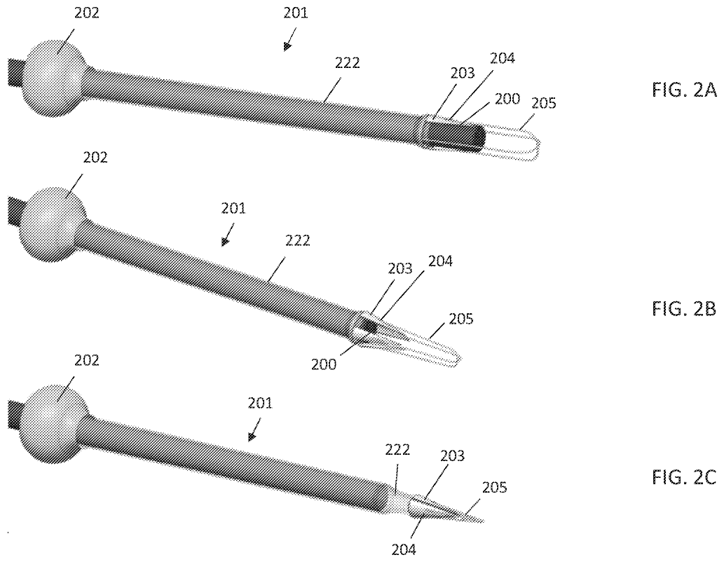

Referring to FIGS. 2A-2C, an over the scope feeding tube 201 can include an elongate tubular portion 222 made from a thin stretchable material, such as medical grade polymer (e.g., silicone). The tubular portion 222 can be configured to stretch around (and attach/conform to) the distal end of a scope 200. The elongate tubular portion 222 can be shorter than a traditional feeding tube, such as between 30-35 cm long. The proximal end of the tubular portion 222 can include a soft stop knob 202 therearound. Additionally, the feeding tube 201 can have a plurality (e.g., 2-10, such as 4) of triangular spikes 203 extending from the distal end thereof. The spikes 203 can be made of a medical grade metal or polymer, such as silicone. The material of the spikes 203 can be harder and/or stiffer than the material of the tubular portion 222. In some embodiments, the proximal ends of the triangular spikes 203 can be embedded in the tubular portion 222 for attachment thereto. The spikes 203 can be 2-6 cm long, such as approximately 4 cm long. Each spike 203 can include a wire 204 extending axially therealong (e.g., centrally through or radially inwards or outwards of the spike 203). The wires 204 can extend distally from each of the spikes 203 and can be formed into distal loops 205 (e.g., either via connection of a plurality of different wires 204 or via a continuous wire looped from one spike 203 to another). If there are a plurality of loops 205, the loops 205 can overlap at a central distal point. Further, the loops 205 can be approximately 8-15 cm long, such as approximately 10 cm long. In some embodiments, the wires 204 can extend at least partially proximally into the tubular portion 222 to provide tensile strength to the spikes 203. The wires 204 can be less than 2 mm in diameter, such as less than 1 mm in diameter, such as less than ½ mm in diameter, such as approximately ⅓ mm in diameter.

As shown in FIGS. 2A-2C, the feeding tube 201 can have a plurality of positions (retracted, partially extended, and fully extended) relative to the scope 200. In FIG. 2A, the feeding tube 201 is in a retracted position whereby the distal tips of the spikes 203 align with the distal tip of the scope 200. In FIG. 2B, the feeding tube 201 is in a partially extended position whereby the tube 201 has been moved distally relative to the scope 200 and the distal tips of the spikes 203 have begun to converge together form a conical distal edge. Finally, in FIG. 2C, the feeding tube 201 is in a fully extended position whereby the tube 201 has been moved distally enough relative to the scope 201 that the spikes 203 have fully converged, forming a sharp distal tip.

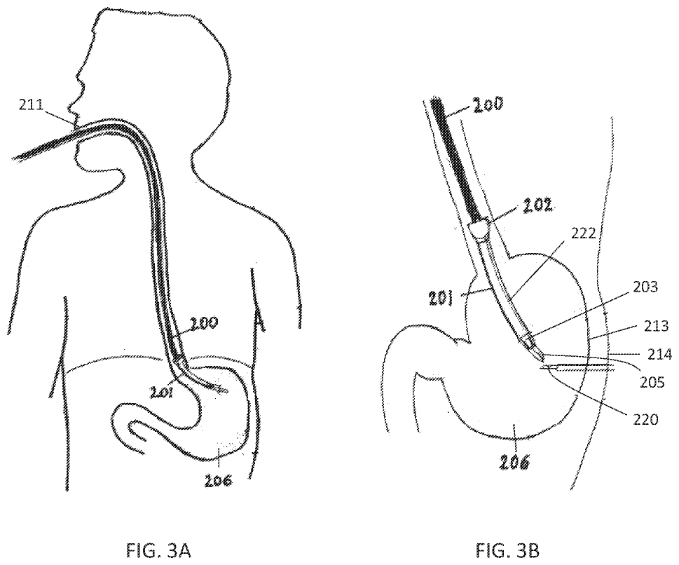

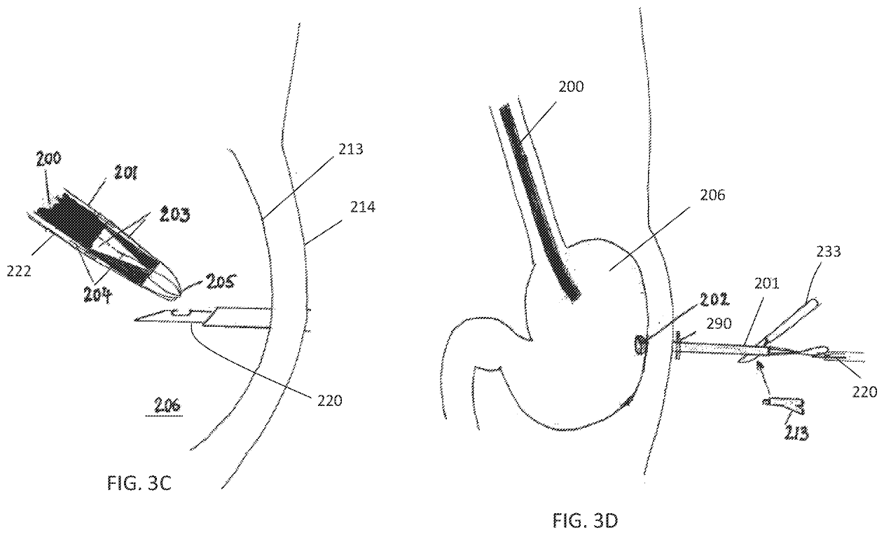

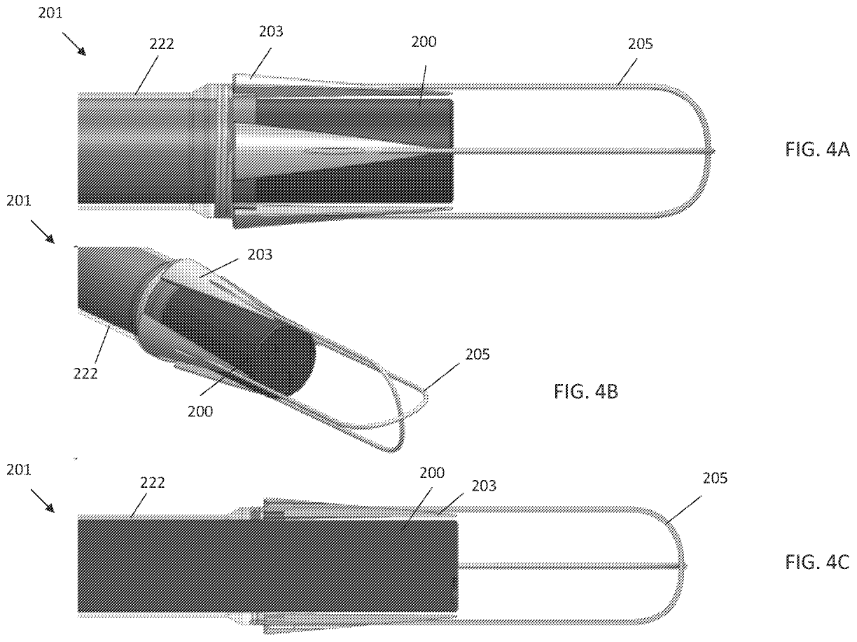

Referring to FIGS. 3A-3D, in use, the feeding tube 201 can be stretched around the distal end of the scope 200. The scope 200 and feeding 201 can then be inserted into through the mouth 211 and into the stomach 206, as shown in FIG. 3A. When the feeding tube 201 is inserted over the scope 200, it can be in the retracted position. Close-ups of the retracted position are shown in FIGS. 4A-4C. Advantageously, the distal tips of the spikes 203 in the retracted position can align with or be proximal to the distal end of the scope 200 and can be radially flush with the distal end of the scope 200, thereby ensuring that the spikes 203 do not engage with the anatomy (e.g., the esophagus) during delivery of the scope 200. Additionally, the scope 200 can still advantageously image through the open loops 205 of the feeding tube 201 when the feeding tube 201 is in the retracted position. Once the scope 200 and feeding tube 201 are in the stomach 206, the stomach 206 can be distended with air, the abdominal wall 114 transilluminated, the external abdominal surface marked and disinfected, and the area anesthetized.

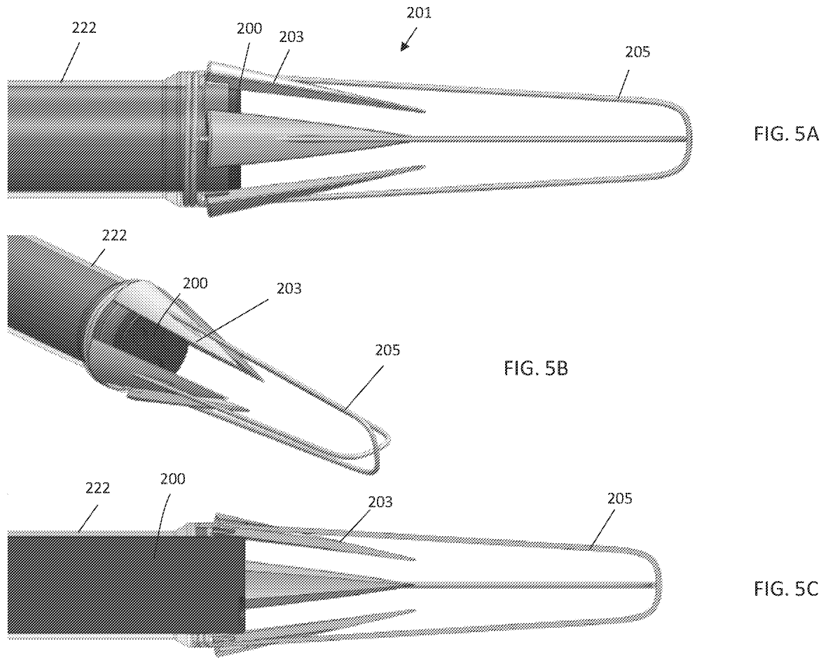

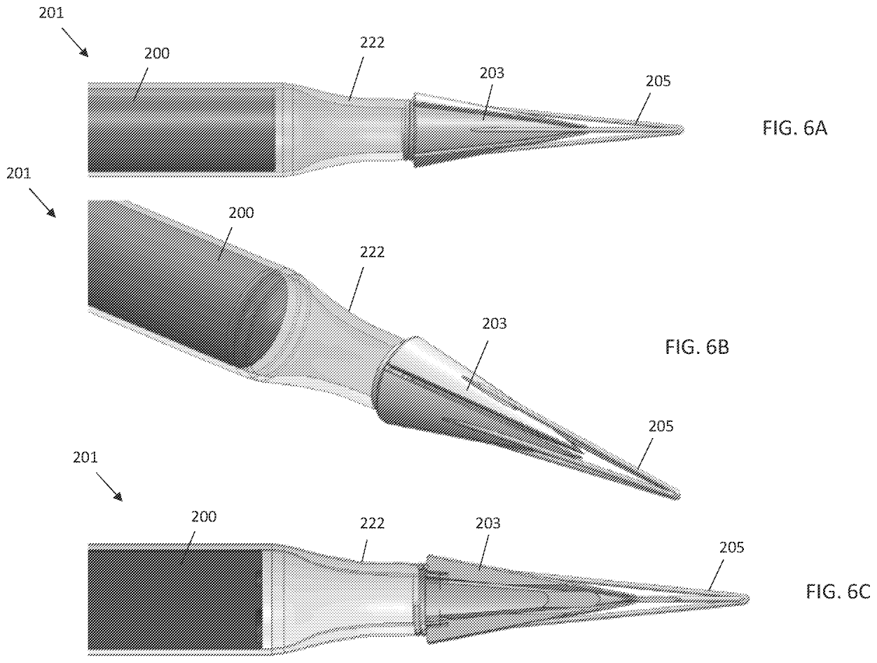

Referring back to FIGS. 3B-3C (FIG. 3C is a close-up of FIG. 3B), a large bore needle 220 can be inserted through the abdominal wall 214 and the gastric wall 213 and into the lumen of the stomach 206. The large bore needle 220 can then be used to grab onto one or more of the loops 205 of the feeding tube 201 while the feeding tube 201 is in the stomach 206. After attaching to one or more of the loops 205, the large bore needle 220 can be pulled distally out through the gastric and abdominal walls 213, 214, causing the feeding tube 201 to be pulled off of the scope 200. By pulling the feeding tube 201 off of the scope 200, the large bore needle 220 can cause the feeding tube 201 to move from the retracted position (of FIGS. 4A-4C) to the partially extended and extended positions. Close-ups of the partially extended and extended positions are shown in FIGS. 5A-5C and 6A-6C, respectively. As shown in FIGS. 5A-5C, when the feeding tube 201 is in the partially extended configuration, the loops 205 can begin to elongate, and the tips of the spikes 203 can extend distally beyond the scope 200 and begin to converge together into a conical distal edge. As shown in FIGS. 6A-6C, when the feeding tube 201 is in the fully extended configuration, the loops 205 can be collapsed into a point, and the spikes 203 can be entirely distal to the scope 200 with the spikes 203 converged into a sharp conical distal tip. The sharp conical distal tip can help penetrate the gastric and abdominal walls 213, 214 as the feeding tube 201 is pulled therethrough. As is further shown in FIGS. 6A-6C, the elongate tube 222 can shrink back to its original diameter after it is removed from its stretched configuration around the scope 200.

Referring back to FIG. 3D, the tube 201 can then be pulled completely out of the stomach 206 except for the soft stop knob 202, which can be used to hold the proximal end of the feeding tube 201 in the stomach 206. The excess feeding tube 201 can then be cut with a cutting instrument 233, and a bolster 290 and feeding adapter 213 can be attached to enable food to be provided directly to the stomach 206 through the feeding tube 201.

Advantageously, the tubular portion 222 can be configured to stretch around a distal end of a scope while still resuming its original shape after removal from the scope 200 so as to serve the purpose of a feeding tube. Additionally, the elongate tubular portion 222 can advantageously be shorter than a traditional tube (because it does not need to extend all the way from the mouth through the abdominal wall during delivery). Further, the method of delivering the feeding tube 201 described herein can advantageously be simpler and take less time than delivery of standard feeding tube. Finally, the feeding tube 201 can enable visualization of the stomach with the endoscope 200 throughout the delivery process (i.e., without having to reintubate to inspect the placement of the tube 201).

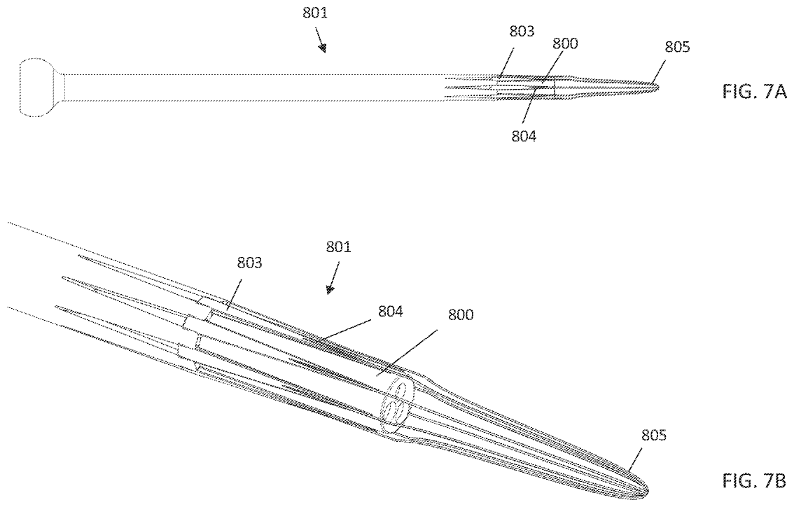

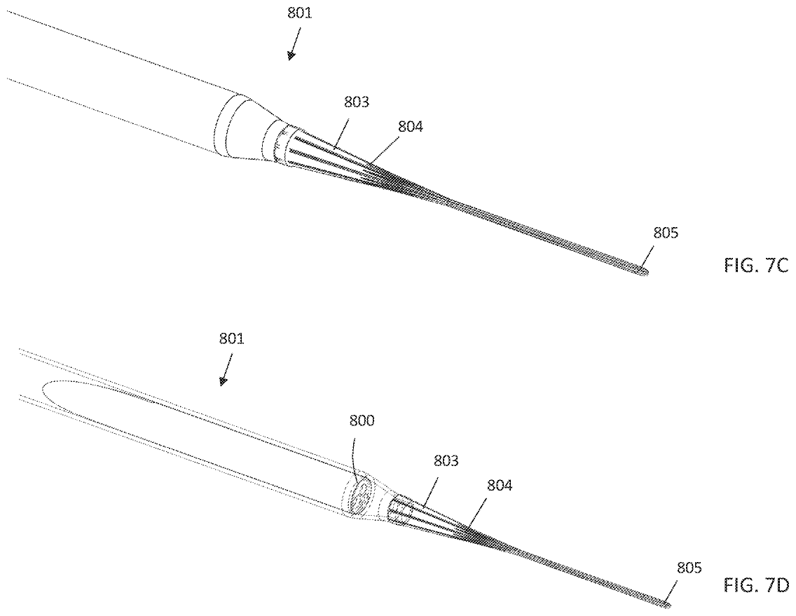

Another embodiment of a feeding tube 801 is shown in FIGS. 7A-7D (where FIGS. 7A-7B show the retracted position relative to the scope 800 and FIGS. 7C-7D show the extended configuration relative to the scope 800). The feeding tube 801 is similar to tube 201 except that tube 801 includes more spikes 803 (e.g., 6-12, such as 8) and corresponding wires 804 (and thus more loops 805).

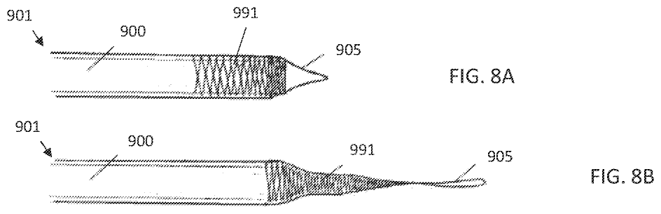

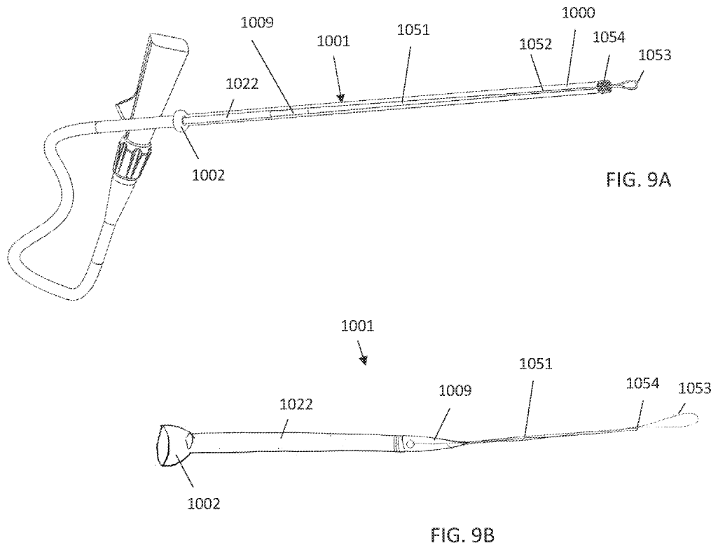

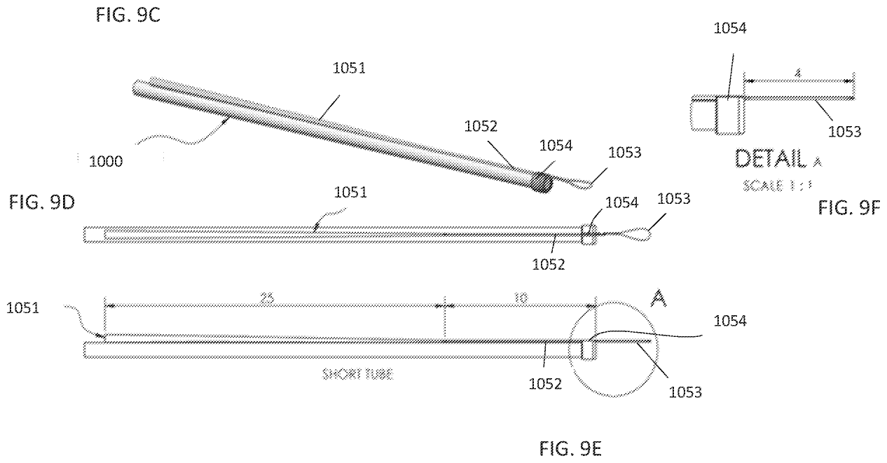

Another embodiment of a feeding tube 901 is shown in FIGS. 8A-8B (where FIG. 8A shows the retracted position relative to the scope 900 and FIG. 9B shows the extended configuration elative to the scope 900). The feeding tube 901 is similar to tube 201 except that the spikes have been replaced with a collapsible mesh tubing 991 (attached to one or more distal loops 905). The mesh tubing 991 may advantageously funnel into a cone shape during delivery (e.g., when being pulled distally out of the stomach and through the gastric and abdominal walls).

Another embodiment of an exemplary feeding tube 1001 is shown in FIGS. 9A-9F. The feeding tube 1001 is configured to run parallel to and along side the scope 1000. The feeding tube 1001 includes an elongate tubular portion 1022 (similar to tubular portion 222 described with respect to FIGS. 2A-2D) having a soft stop knob 1002 at the proximal end thereof and a conical distal tip 1009. Additionally, the feeding tube 1001 includes a tapered section 1051 extending distally from the elongate tubular portion 1022 (e.g., from the conical tip 1009). For example, the tapered section 1051 can have a diameter of 9 mm at the proximal end thereof and a diameter of 2 mm at the distal end 1052. In some embodiments, a distal end 1052 of the tapered section 1051 can have a segment (e.g., a segment having a length of 5-5 cm, such as 10 cm) of constant diameter, such as a 2 mm diameter. The tapered section 1051 can, for example (and as shown in FIG. 9B), include a wire embedded within a plastic layer, such as a silicone layer. Finally, a wire can extend from the distal end 1052 of the tapered section 1051 and can be formed into a loop 1053 distal to the scope 1000.

The feeding tube 1001 can be attached to the scope 1000 with a releasable distal attachment mechanism 1054 that connects the distal end 1052 of the tapered portion 1051 to the distal tip of the scope 1000. The distal attachment mechanism 1054 can be the only attachment location for the feeding tube 1001 along the length of the scope 1000.

In the embodiment shown in FIGS. 9A-9F, the feeding tube 1001 can be configured to be less than 80 cm in length, such as less than 60 cm in length, such as less than 50 cm in length. In this embodiment, for example, the tapered section 1051 can extend approximately 30-40 cm (such as 35 cm), and the loop 1053 can extend 2-8 cm (such as 4 cm). This “short” feeding tube 1001 can be used, for example, where there is no blockage along the upper GI tract and the tube 1001 can easily be pulled alongside the scope 1000 into the gastric cavity. For example, the “short” feeding tube 1001 might be used for patients with difficulty swallowing that are neurogenic (e.g., after a stroke). Advantageously, the short tube 1001 can enable visualization of the stomach with the endoscope 1000 throughout the delivery process (i.e., without having to reintubate to inspect the placement of the short tube 1001).

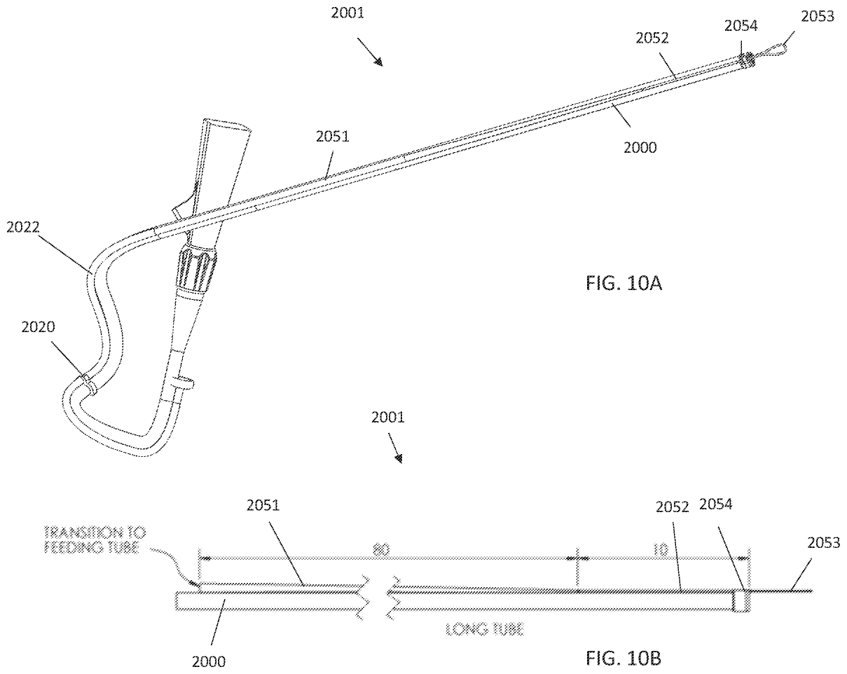

FIGS. 10A-10B show a feeding tube 2001 that is similar to feeding tube 1001 except that the tube 2001 is a “long” feeding tube rather than a “short” feeding tube. That is, the feeding tube 2001 can be configured to be longer than 80 cm in length, such as longer than 90 cm, such as longer than 100 cm. The long feeding tube 2001 can be attached to the scope 2000 (e.g., near the scope handle) with a proximal attachment mechanism 2020 so as to prevent the tube 2001 from dangling around outside the body during use. In this embodiment, for example, the tapered section 2051 can extend approximately 80-100 cm (such as 90 cm), and the loop 2053 can extend 2-8 cm (such as 4 cm). This “long” feeding tube 2001 can be used, for example, where there is an obstruction or blockage along the upper GI tract. In this case, the tapered portion 2051 can extend along the scope 2000 as the scope 2000 is inserted into the patient through the mouth while the larger elongate tubular portion 2022 can remain outside of the mouth. As described below, the elongate tubular portion 2022 can be pulled through the upper GI tract after the scope 2000 is removed, thereby ensuring that there is enough space within the upper GI tract for the tubular portion 2022 to pass therethrough.

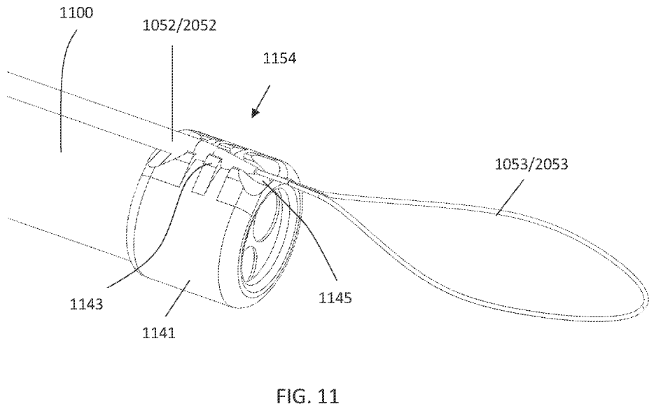

Exemplary attachment mechanisms for the feeding tubes 1001/2001 (e.g., that can be used as attachment mechanisms 1054/2054) are shown in FIGS. 11-13B. Referring to FIG. 11, for example, an attachment mechanism 1154 includes an annular ring 1141 configured to slide over the distal end of the scope 1100. The annular ring 1141 further includes a longitudinal channel 1145 in which the distal end 1052/2052 of the tapered section 1051/2051 can be positioned. The distal end 1052/2052 can be held within the channel 1145 by teeth 1143 (e.g., via a snap or friction fit) that line the channel 1145. To release the distal end 1052/2052 from the attachment mechanism 1154, the loop 1053/2053 can be pulled laterally (e.g., away from the attachment mechanism 1154), causing the distal end 1052/2052 to release from the teeth 1143. This release of the distal end 1052/2052 can correspondingly enable the feeding tube 1001/2001 to disengage from the scope 1100.

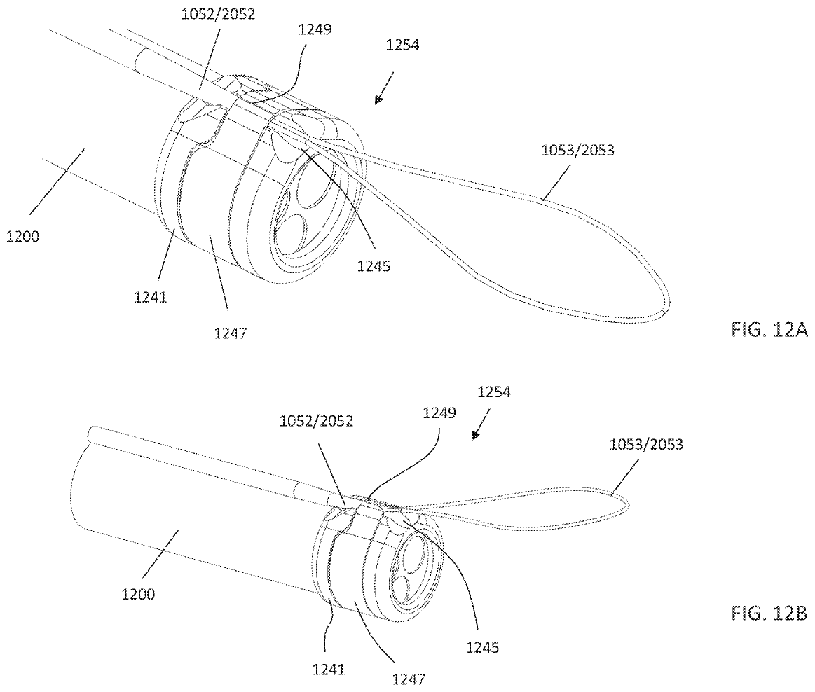

Referring to FIGS. 12A-12B, another exemplary attachment mechanism 1254 includes an annular ring 1241 having an axial channel 1245 therein in which the distal end 1052/2052 of the tapered section 1051/2051 can be positioned. The annular ring 1241 can further include a thin band 1247 extending therearound that can hold the distal end 1052/2052 inside the channel 1245, as shown in FIG. 12A. The thin band 1247 can include a perforation 1249 extending axially therethrough. Further, the perforation 1249 can be circumferentially aligned with the axial channel 1245. As shown in FIG. 12B, to release the distal end 1052/2052 from the attachment mechanism 1254, the loop 1053/2053 can be pulled laterally (e.g., away from the attachment mechanism 1254), causing the distal end 1052/2052 to tear the thin band 1247 at the perforation 1249 and release from the distal end 1052/2052. This release of the distal end 1052/2052 of the tapered section 1051/2051 can correspondingly enable the feeding tube 1001/2001 to disengage from the scope 1200.

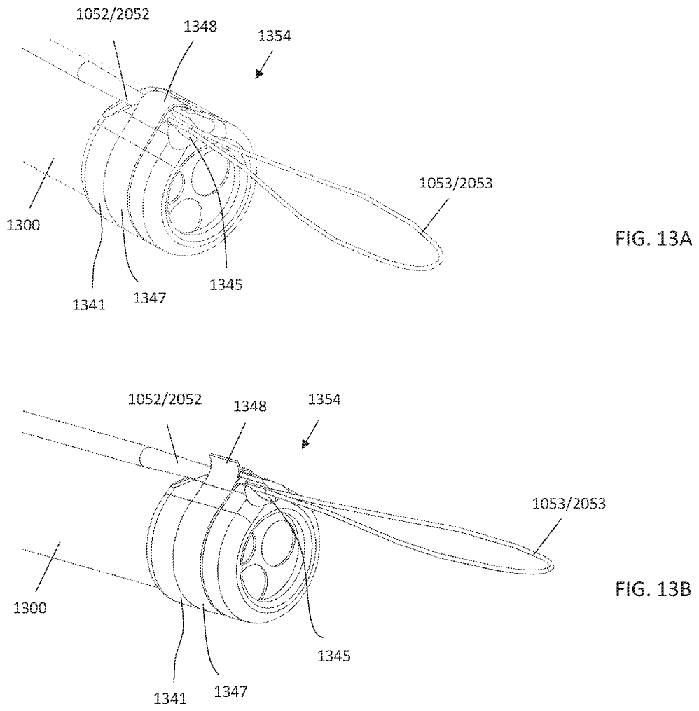

Referring to FIGS. 13A-13B, another exemplary attachment mechanism 1354 can includes an annular ring 1341 having an axial channel 1345 therein in which the distal end 1052/2052 of the tapered section 1051/2051 can be positioned. The annular ring 1341 can further include a thin band 1347 extending therearound that can hold the distal end 1052/2052 inside the channel 1345, as shown in FIG. 13A. The thin band 1347 can include overlapping flaps 1348 (e.g., made of silicone) that can hold together via friction. Further, the overlapping flaps 1348 can be circumferentially aligned with the axial channel 1345. As shown in FIG. 13B, to release the distal end 1052/2052 from the attachment mechanism 1354, the loop 1053/2053 can be pulled laterally (e.g., away from the attachment mechanism 1354), causing the distal end 1052/2052 to open the flaps 1348 and release the distal end 1052/2052. This release of the distal end 1052/2052 can correspondingly enable the feeding tube 1001/2001 to disengage from the scope 1300.

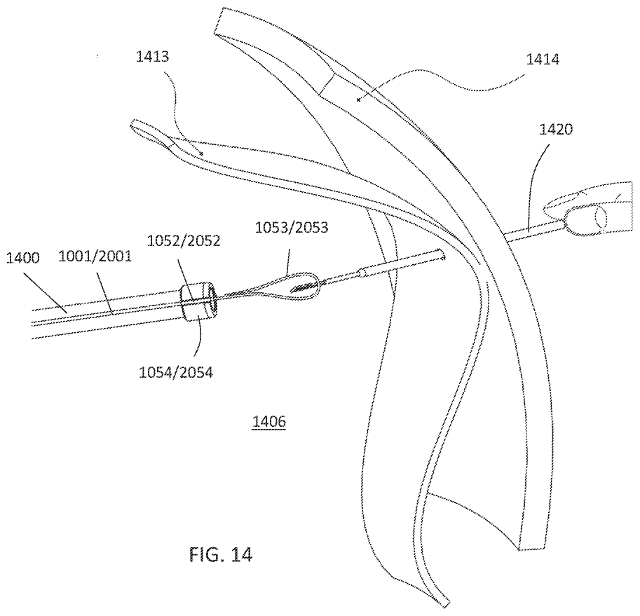

Referring to FIG. 14, in use, the feeding tube 1001/2001 can be attached to the distal end of the scope 1400 with attachment mechanism 1054/2054. The scope 1400 and attached feeding tube 1001/2001 can then be passed through the mouth and into the stomach 1406. Once the scope 1400 and feeding tube 1001/2001 are in the stomach 1406, the stomach 1406 can be distended with air, the abdominal wall 1414 transilluminated, the external abdominal surface marked and disinfected, and the area anesthetized.

A large bore needle 1420 can then be inserted through the abdominal wall 1414 and the gastric wall 1413 and into the lumen of the stomach 1406. The large bore needle 1420 can be used to grab onto the loop 1053/2053 of the feeding tube 1001/2001. After attaching to the loop 1053/2053, the large bore needle 1420 can pull laterally on the loop 1053/2053 to disengage the distal end 1052/2052 from the scope 1400 (via release of the attachment mechanism 1054/2054). Once disengaged, the scope 1400 can remain in place (e.g., if the short feeding tube 1001 is being used) or can be removed from the GI tract to create additional room for delivery of the feeding tube (e.g., if the long feeding tube 2001 is being used). The feeding tube 1001/2001 can then be pulled distally (i.e., partially out of the stomach 1406) and attached to the gastric/abdominal walls 1413/1414 similarly to as described with respect to feeding tube 201.

Similar to feeding tube 201, the method of delivering the feeding tube 1001/2001 can advantageously be simpler and take less time than delivery of a standard feeding tube, eliminating the use of wires and/or snares and also reducing the number of personal required to deliver the feeding tube.

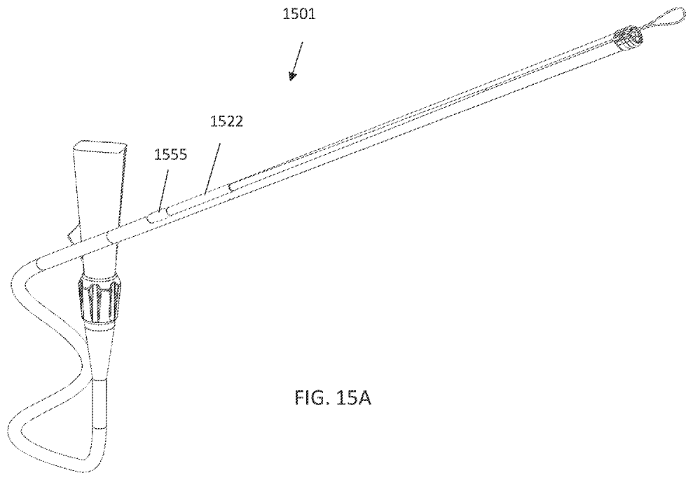

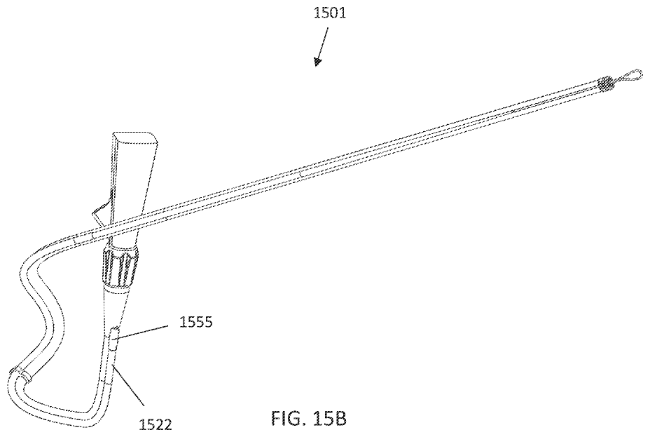

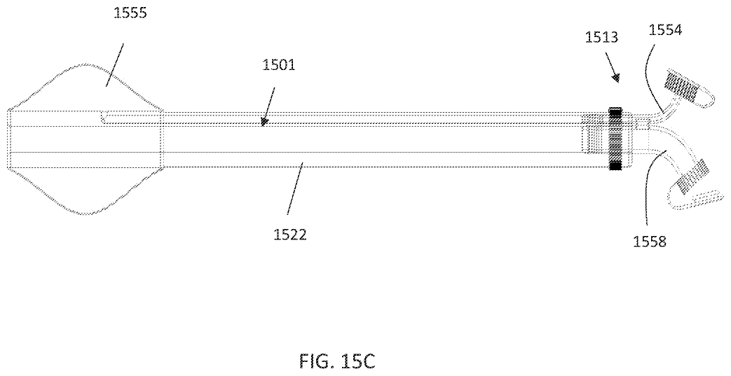

Another embodiment of a feeding tube 1501 is shown in FIGS. 15A-15C. The feeding tube 1501 is similar to the feeding tubes 1001/2001 (where 15A shows the short version and 15B the long version) except that the soft knob is replaced with an inflatable balloon 1555 at the proximal end of the elongate tube 1522. The inflatable balloon 1555 can advantageously make removal and/or replacement of the feeding tube easier than a soft knob. The feeding tube 1501 after delivery/placement through the gastric and abdominal walls is shown in FIG. 15C. Here, the distal end of the tube 1522 has been removed and an adapter 1513 inserted into the distal end. The adapter 1513 can include an inflation lumen 1554 (shown closed) to enable inflation/deflation of the balloon 1555 and a feeding lumen 1558 (shown open) to enable delivery of food directly to the stomach. In this embodiment, the two lumens 1554, 1558 can be parallel with one another within the adapter 1513.

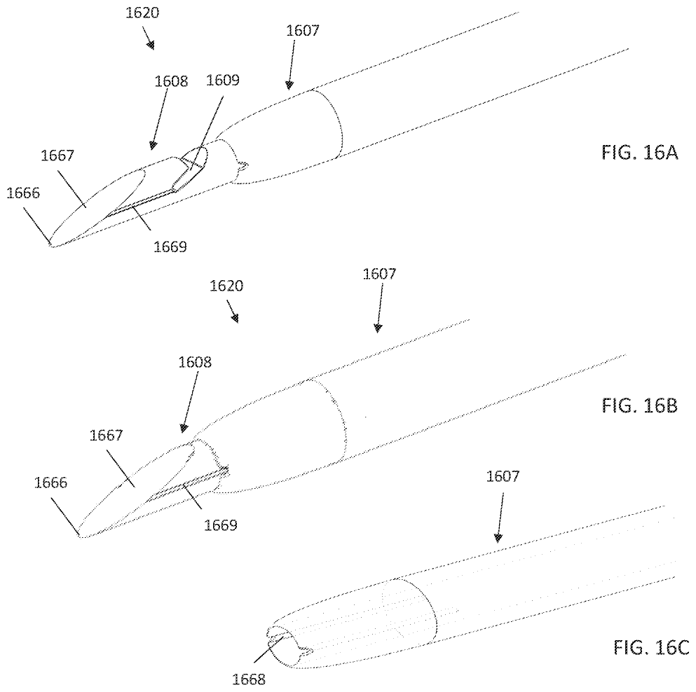

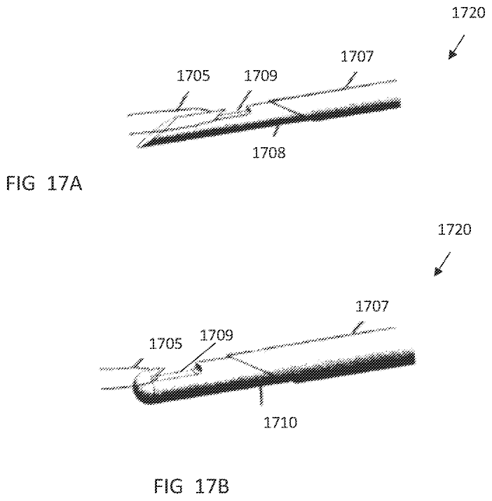

Exemplary large bore needles that can be used to deliver the feeding tubes described herein are shown in FIGS. 16A-16C and 17A-17B (e.g., that can be used as large bore needles 220 or 1420). In one embodiment, shown in FIG. 16A-16C, a large bore needle 1620 can include a tapered outer sheath 1607 and a removable inner stylet 1608. The inner stylet 1608 can include an angled cutting surface 1667 ending in a sharp tip 1666 that can be configured to pierce through the abdominal and gastric walls. Further, the inner stylet 1608 can include a groove 1609 therein configured to grab a distal loop of a feeding tube as described herein. The groove 1609 can be transverse to the longitudinal axis of the sharp inner stylet 1608. Further, the groove 1609 can be slanted towards the sharp tip 1666 (e.g., so as to be substantially parallel to the cutting surface 1667). Further, the inner stylet 1608 can include two axially extending channels 1669 in the outer surface of the stylet 1608 that connect the groove 1609 with the cutting surface 1667. The channels 1669 can advantageously further house the wire loop therein. As shown in FIG. 16C, the outer sheath 1607 can include corresponding channels 1668 in the inner surface thereof that are configured to align with the channels 1669 of the inner stylet 1608 to better house the wire loop. In use, the outer sheath 1607 can advantageously cover the groove 1609 as the stylet 1608 cuts through the gastric and abdominal walls to prevent the groove 1609 from catching during insertion. Once inserted into the stomach, the outer sheath 1607 can be pulled back and the loop of the feeding tube placed within the groove 1609 and along the channels 1669/1668. The outer sheath 1607 can then be pushed back over the groove 1609 and some or all of the channels 1669/1669 (as shown in FIG. 16B) to trap the loop therein and ensure that the feeding tube does not separate from the large bore needle 1620 as it is pulled through the gastric and abdominal walls.

Another embodiment of a large bore needle 1720 is shown in FIGS. 17A-17B. The large bore needle 1720 can be similar to the large bore needle 1620 except that the sharp inner stylet 1708 can be interchangeable with an atraumatic stylet hook 1710. Similar to the large bore needle 1620, the large bore needle 1720 can include an outer sheath 1707. The outer sheath 1707 in this embodiment can be configured to cover the groove 1709 in either the sharp inner stylet 1720 or the stylet hook 1710.

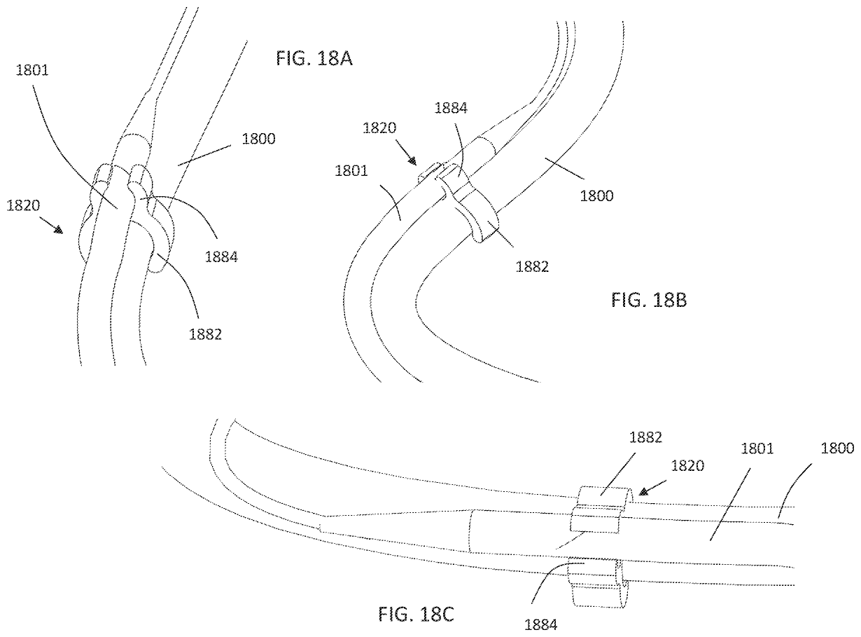

An exemplary proximal attachment mechanism 1820 (e.g., for use as attachment mechanism 2220 for long tube 2001 or as the proximal attachment mechanism for the long tube shown in FIG. 15B) is shown in FIGS. 18A-18C. The attachment mechanism 1820 includes two continuous lobes 1882, 1884 configured to snap around the scope 1800 and the feeding tube 1801, respectively.

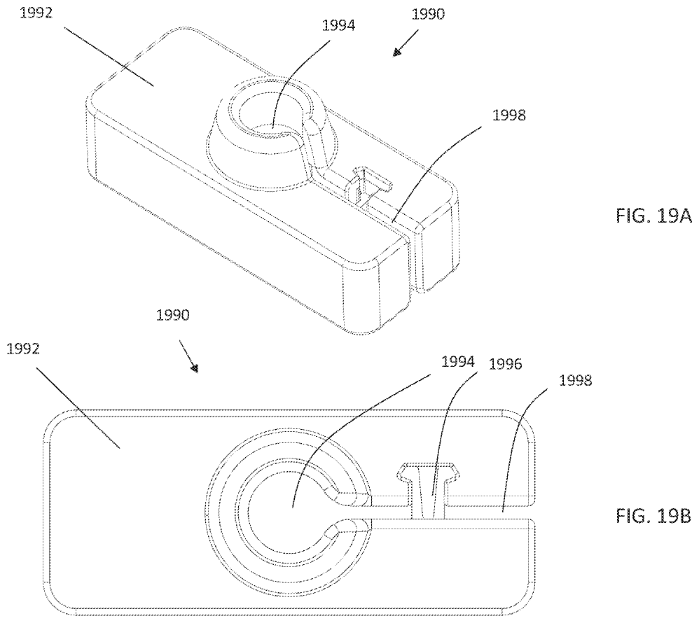

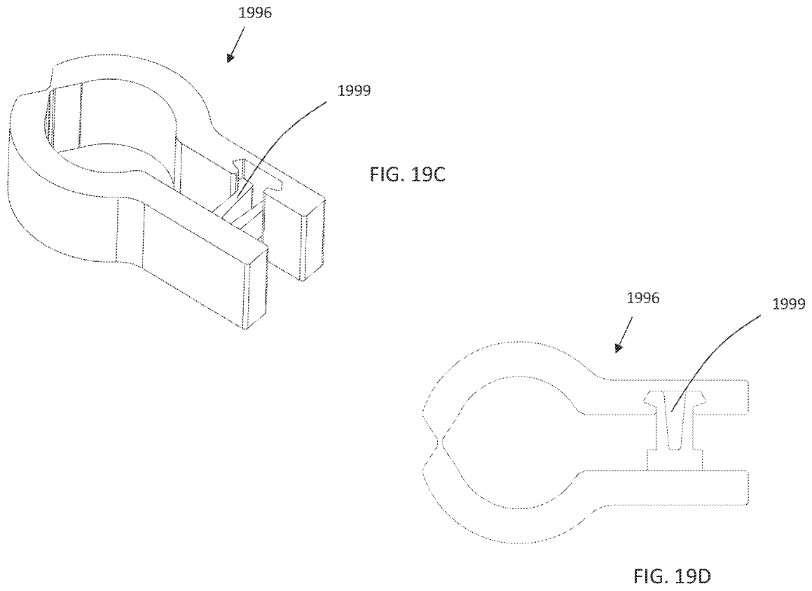

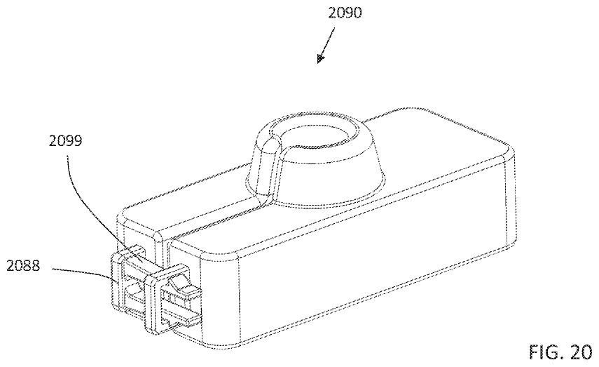

Any of the feeding tubes described herein can be used with an external bolster configured to sit against the abdominal wall to hold the feeding tube in place (in conjunction with the external soft knob or inflatable balloon). Exemplary external bolsters are shown in FIGS. 19A-19D and 20. Referring to FIGS. 19A-19D, an external bolster 1990 can be configured to slide laterally over the feeding tube (i.e., rather than having to load the bolster from the distal end of the feeding tube). The bolster 1990 can include an outer housing 1992 having a central circumferential channel 1994 configured to be positioned around the feeding tube. Further, a lateral slit 1998 can extend through the housing 1992 from the circumferential channel 1994 through the side of the housing 1992. The lateral slit 1998 can enable passage of the feeding tube therethrough when attaching the bolster 1990. The outer housing 1992 can be made of a soft material, such as silicone. The bolster 1990 can further include a core 1996 positioned therein. The core 1996 can extend around the circumferential channel 1994 and on both sides of the slit 1998. Further, the core 1996 can include an internal locking mechanism 1999, such as a snap, configured to lock the slit 1998 closed. The core 1996 can be made of a material (e.g., a polymer) that is harder than the material of the outer housing 1996 so as to better support the slit 1998 and the locking mechanism 1999.

Another exemplary external bolster 2090 is shown in FIG. 20. The bolster 2090 is similar to bolster 1990 except that the bolster 2090 includes an external locking mechanism configured to lock via sliding of the clip portion 2099 into the locking rungs 2088.

Advantageously, the bolsters 1990, 2090 can be easier, less cumbersome, and potentially less painful to clip on and off of patients than traditional bolsters.

It should be understood that features described with respect to one embodiment can be substituted for or combined with features described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. A gastronomy tube, comprising:
an elongate tubular portion configured to extend along a length of an endoscope and to reversibly attach to the endoscope;
a stop at a proximal end of the elongate tubular portion; and
at least one wire loop at a distal end of the elongate tubular portion and configured to extend distally from a distal end of the endoscope when the elongate tubular portion is attached to the endoscope.

2-9. (canceled)

10. The gastronomy tube of claim 1, wherein the at least one wire loop is 8-15 cm long.

11. The gastronomy tube of claim 1, wherein the at least one wire loop comprises a wire having a diameter of less than 1 mm.

12. (canceled)

13. The gastronomy tube of claim 1, further comprising a tapered section that tapers in diameter from the elongate tubular portion towards the at least one wire loop.

14. The gastronomy tube of claim 13, further comprising a distal attachment mechanism configured to reversibly attach the elongate tubular portion to the endoscope by engaging with the tapered section.

15. The gastronomy tube of claim 14, wherein the distal attachment mechanism comprises an annular ring configured to fit over a distal tip of the endoscope and to hold the tapered section therein.

16. The gastronomy tube of claim 15, wherein the distal attachment mechanism further comprises a plurality of teeth, perforations, or flaps configured to release the tapered section from the endoscope upon lateral movement of the at least one wire loop.

17-18. (canceled)

19. A method of delivering a gastronomy tube, comprising:
attaching a gastronomy tube to an endoscope;
passing the endoscope and attached gastronomy tube through an oral cavity and into a stomach;
inserting a large bore needle through an abdominal wall, a gastric wall, and into a lumen of the stomach;
grabbing a distal loop of the gastronomy tube with the large bore needle;
pulling the distal loop distally such that the gastronomy tube is pulled distally out of the stomach; and
attaching the gastronomy tube to the abdominal wall and the gastric wall.

20. The method of claim 19, wherein pulling the distal loop distally disengages the gastronomy tube from the endoscope.

21-23. (canceled)

24. The method of claim 19, wherein attaching the gastronomy tube to the endoscope comprises placing an annular attachment mechanism around a distal tip of the endoscope and extending the gastronomy tube parallel to the endoscope.

25. The method of claim 24, further comprising disengaging the gastronomy tube from the endoscope by pulling laterally on the distal loop.

26. The method of claim 25, wherein the endoscope is left in the stomach during the step of pulling the distal loop distally.

27-29. (canceled)

30. The method of claim 19, wherein the large bore needle comprises an inner stylet and an outer sheath, and wherein grabbing the distal loop of the gastronomy tube with the large bore needle comprises grabbing the distal loop with a groove in the inner stylet.

31. The method of claim 30, further comprising pushing the outer sheath over the distal loop when the distal loop is positioned in the groove prior to the step of pulling the distal loop distally.

32-33. (canceled)

34. A large bore needle, comprising:
an inner stylet having a central longitudinal axis and a sharp distal tip, the inner stylet comprising a groove in an outer surface thereof, the groove extending transverse to the central longitudinal axis and configured to hold a wire therein; and
an outer sheath positioned over the inner stylet, the sheath configured to slide over and reversibly lock the wire inside of the groove.

35. The large bore needle of claim 34, wherein the inner stylet further comprises a plurality of channels extending from the groove towards the sharp distal tip, the plurality of channels further configured to hold the wire therein.

36. The large bore needle of claim 35, wherein the plurality of channels extend parallel to the central longitudinal axis.

37. The large bore needle of claim 35, wherein the outer sheath further comprises a plurality of axial channels on an inner circumference thereof, the axial channels configured to align with the channels of the inner stylet to hold the wire therein.

38. (canceled)

39. A bolster for attaching a feeding tube to a patient, the bolster comprising:
a housing;
a bore through the housing;
a slit through the housing, the slit extending from a lateral surface of the housing to the bore; and
a locking mechanism configured to lock the slit together when a feeding tube is positioned within the bore.

40-42. (canceled)
